(19) United States
(12) Patent Application Publication
(10) Pub. No.: US 2013/0058957 A1
(43) Pub. Date: Mar. 7, 2013

(54) DENDRITIC CELL IMMUNORECEPTOR AGONIST

(71) Applicants: Yoichiro Iwakura, Bunkyo-ku (JP); Noriyuki Fujikado, Bunkyo-ku (JP); Guangyu Ma, Bunkyo-Ku (JP)

(72) Inventors: Yoichiro Iwakura, Bunkyo-ku (JP); Noriyuki Fujikado, Bunkyo-ku (JP); Guangyu Ma, Bunkyo-Ku (JP)

(73) Assignee: THE UNIVERSITY OF TOKYO, Tokyo (JP)

(21) Appl. No.: 13/580,689

(22) PCT Filed: Feb. 23, 2011
(86) PCT No.: PCT/JP2011/053980

§ 371 Date: Nov. 20, 2012

(30) Foreign Application Priority Data
Feb. 23, 2010 (JP) 2010-037204

(51) Int. Cl. A61K 39/395; G01N 33/566; A61P 35/00; A61K 31/737; A61P 29/00; A61P 19/08; C08B 37/00; C07K 16/28

(52) U.S. Cl. 424/172.1; 536/54; 435/7.24; 530/389.8; 530/388.22; 514/54

(57) ABSTRACT

An object of the present invention is to find a ligand for a DCIR and to search for an agonist and an antagonist for the DCIR.

Specifically, disclosed are: a dendritic cell immunoreceptor agonist containing keratan sulfate-II (KS-II) as an active ingredient; an antibody against dendritic cell immunoreceptor, having a keratan sulfate-II-like dendritic cell immunoreceptor agonism; and an antibody against a dendritic cell immunoreceptor, having a keratan sulfate-II inhibitory dendritic cell immunoreceptor antagonism.

### TECHNICAL FIELD

The present invention relates to a ligand for a dendritic cell immunoreceptor (DCIR), an anti-DCIR antibody and use thereof.

### BACKGROUND ART

Dendritic cells (DCs) are main antigen-presenting cells (APCs) and play a central role in immune system regulation. In recent years, some C-type lectin receptors (CLRs) were characterized that they are expressed on the surfaces of dendritic cells. MMR (CD206) and DEC-205 (CD205), which are members of type I CLR, have a plurality of calcium-dependent extracellular carbohydrate recognition domains (CRDs) at the N-terminal. A second family of the CLR expressed on a dendritic cell is type II protein having a single CRD at the C-terminal, and the family includes DC-SIGN (CD209), Langerin (CD207), CLEC-1, Dectin-1 (β-GR), Dectin-2, DLEC, and DCIR.

DCIR is also called LLIR and is type II membrane protein mainly expressed on human and mouse dendritic cells. This molecule has one carbohydrate recognition domain (CRD) in an extracellular domain and a consensus ITIM in an intracellular domain. Since the ITIM transmits an inhibitory signal to cells, the mouse DCIR is suggested to act as an inhibitory receptor and to control the dendritic cell function.

The present inventors previously succeeded in producing DCIR knockout mice and reported that through investigation using the mice, DCIR is involved in development of arthritis and rheumatoid arthritis (Patent Literature 1 and Non-Patent Literature 1).

### PRIOR ART LITERATURE

### Patent Literature

[Patent Literature 1] JP-A-2008-29319
[Patent Literature 2] JP-A-2009-19044

### Non-Patent Literature

[Non-Patent Literature 1] Nature Medicine, Vol. 14, No. 2, pp. 176-180, February 2008

### SUMMARY OF INVENTION

### Problem to be Solved by the Invention

Unfortunately, no endogenous ligand for DCIR was found yet, and therefore the mechanism of action of DCIR in vivo has not been really elucidated. In addition, no DCIR agonist or DCIR antagonist was found at all.

Accordingly, it is an object of the present invention to find a ligand for a DCIR and to search for an agonist and an antagonist for the DCIR and to put them into practical use.

### Means for Solving the Problem

The present inventors variously investigated for finding a ligand for DCIR and, as a result, surprisingly found that keratan sulfate-II (KS-II) is the ligand. The present inventors have further investigated the action by binding of KS-II and DCIR to each other and have found that KS-II controls the functions of osteoblasts and osteoclasts by binding to DCIR, in particular, highly suppresses osteoclast formation and suppresses inflammation. Based on these findings, the inventors further produced antibodies against DCIR and investigated the activity thereof. As a result, the inventors found that anti-DCIR antibodies include not only antibodies KS-II inhibitory DCIR antagonism not also antibodies having KS-II-like DCIR agonism. The inventors have also found that the anti-DCIR antibody having the KS-II-like DCIR agonism has a high osteoclast formation-suppressing activity and a TNF-α production-suppressing activity and is therefore useful as an agent for preventing or treating diseases accompanied by abnormal bone metabolism and inflammatory diseases. Thus, the present invention was accomplished.

That is, the present invention provides a dendritic cell immunoreceptor agonist containing KS-II as an active ingredient.

Furthermore, the present invention provides a method of screening for a dendritic cell immunoreceptor agonist or a dendritic cell immunoreceptor antagonist, including measuring binding affinity of a test substance to a dendritic cell immunoreceptor in the presence of KS-II or using KS-II as a control.

Furthermore, the present invention provides an antibody against a dendritic cell immunoreceptor, having a KS-II-like dendritic cell immunoreceptor agonism, and a pharmaceutical containing the antibody.

Furthermore, the present invention provides an antibody against a dendritic cell immunoreceptor, having a KS-II inhibitory dendritic cell immunoreceptor antagonism, and a pharmaceutical containing the antibody.

Furthermore, the present invention provides KS-II for use in agonizing a dendritic cell immunoreceptor.

Furthermore, the present invention provides use of KS-II for producing a dendritic cell immunoreceptor agonist.

Furthermore, the present invention provides an antibody against a dendritic cell immunoreceptor, having a KS-II-like dendritic cell immunoreceptor agonism, for use in preventing or treating a disease accompanied by abnormal bone metabolism or an inflammatory disease.

Furthermore, the present invention provides use of an antibody against a dendritic cell immunoreceptor, having a KS-II-like dendritic cell immunoreceptor agonism, for producing an agent for preventing or treating a disease accompanied by abnormal bone metabolism or an inflammatory disease.

Furthermore, the present invention provides an antibody against a dendritic cell immunoreceptor, having a KS-II inhibitory dendritic cell immunoreceptor antagonism, for use in a cancer treatment or for immunostimulation.

Furthermore, the present invention provides use of an antibody against a dendritic cell immunoreceptor, having KS-II inhibitory dendritic cell immunoreceptor antagonism, for producing an anticancer agent or an immunostimulant.

Furthermore, the present invention provides a method of agonizing a dendritic cell immunoreceptor, including administering KS-II.

Furthermore, the present invention provides a method of preventing or treating a disease accompanied by abnormal bone metabolism or an inflammatory disease, including administering an antibody against a dendritic cell immunoreceptor, having a KS-II-like dendritic cell immunoreceptor agonism.

Furthermore, the present invention provides a method of preventing or treating a cancer or of immunostimulation, including administering an antibody having a KS-II inhibitory dendritic cell immunoreceptor antagonism.

### Advantageous Effects of Invention

According to the present invention, it was found that KS-II is a ligand for DCIR. KS-II has an osteoclast formation-suppressing activity and the like and is useful as a novel pharmaceutical. Furthermore, among anti-DCIR antibodies, an antibody having a KS-II-like activity has a DCIR agonism and is useful as an agent for preventing or treating a disease accompanied by abnormal bone metabolism, an inflammatory disease or the like. In contrast, an antibody having a KS-II inhibiting activity has a DCIR antagonism and is useful as an anticancer agent, an immunostimulant or the like.

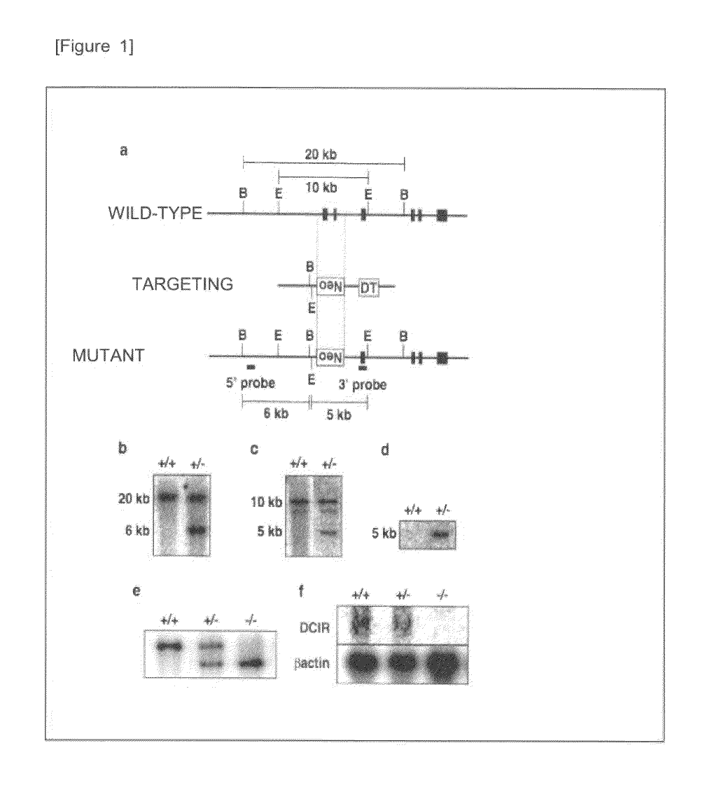

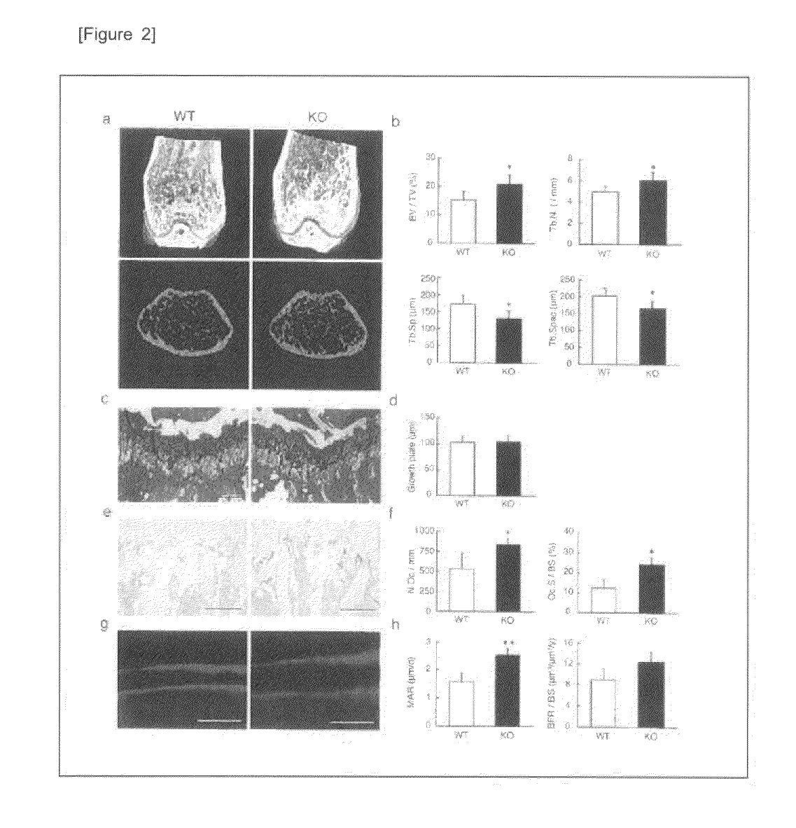

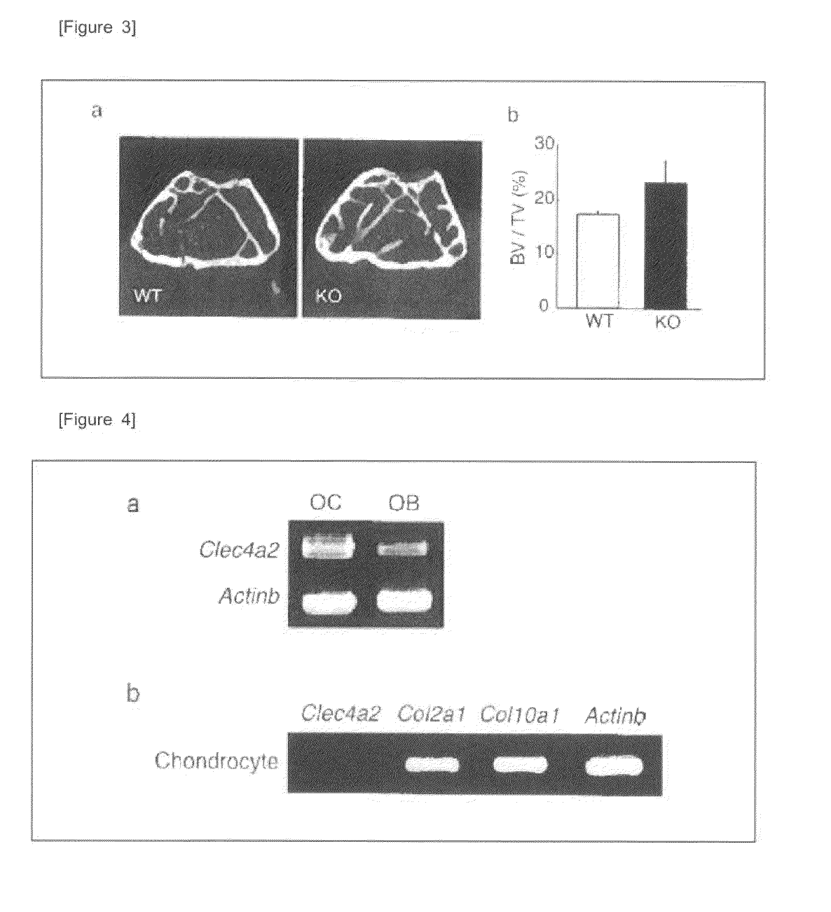

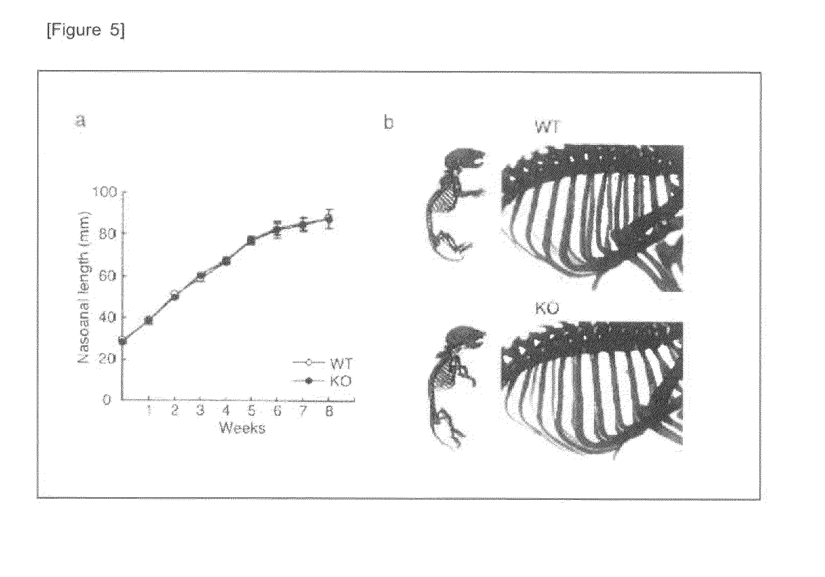

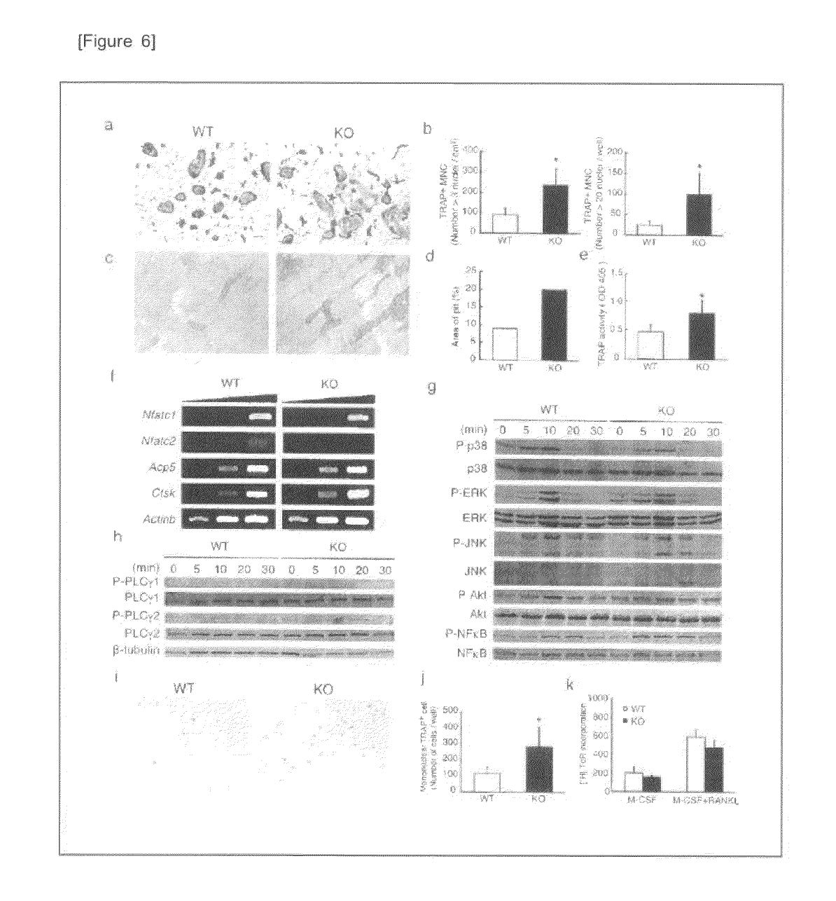

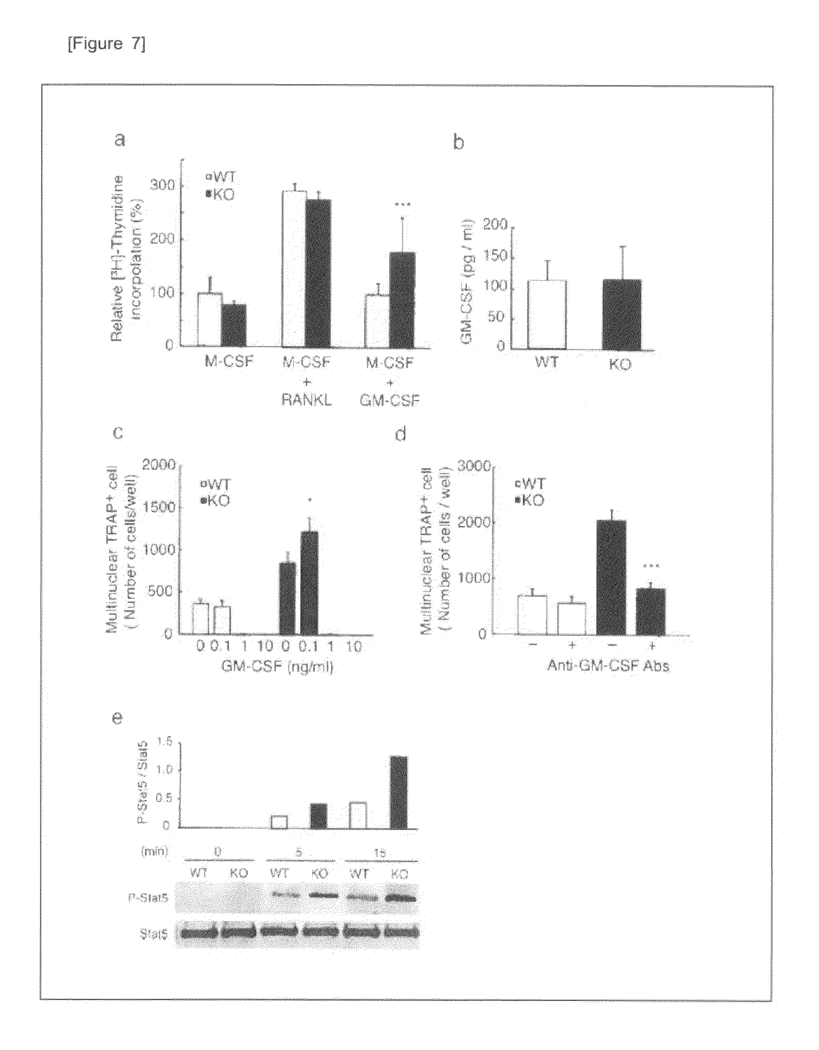

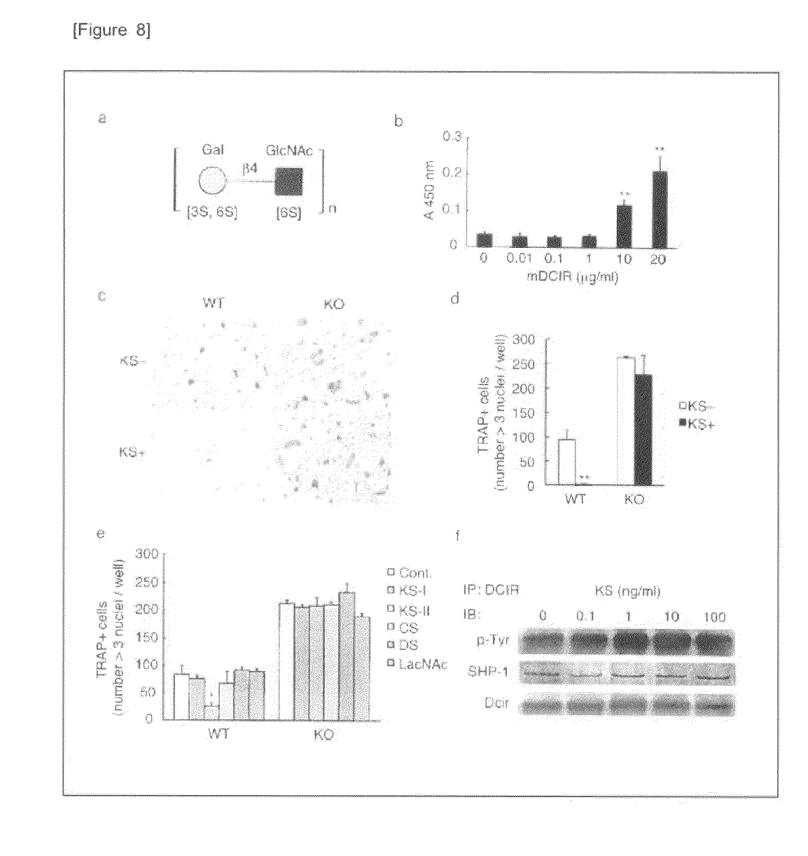

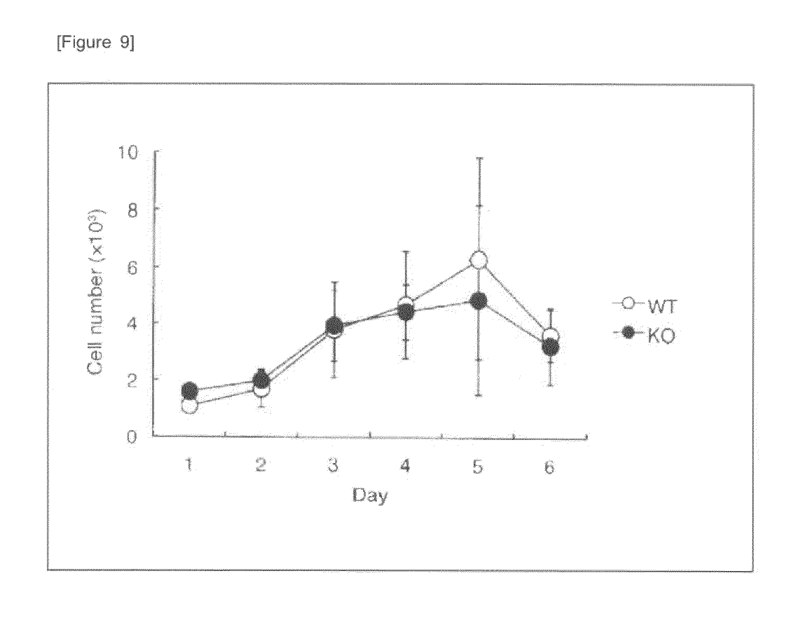

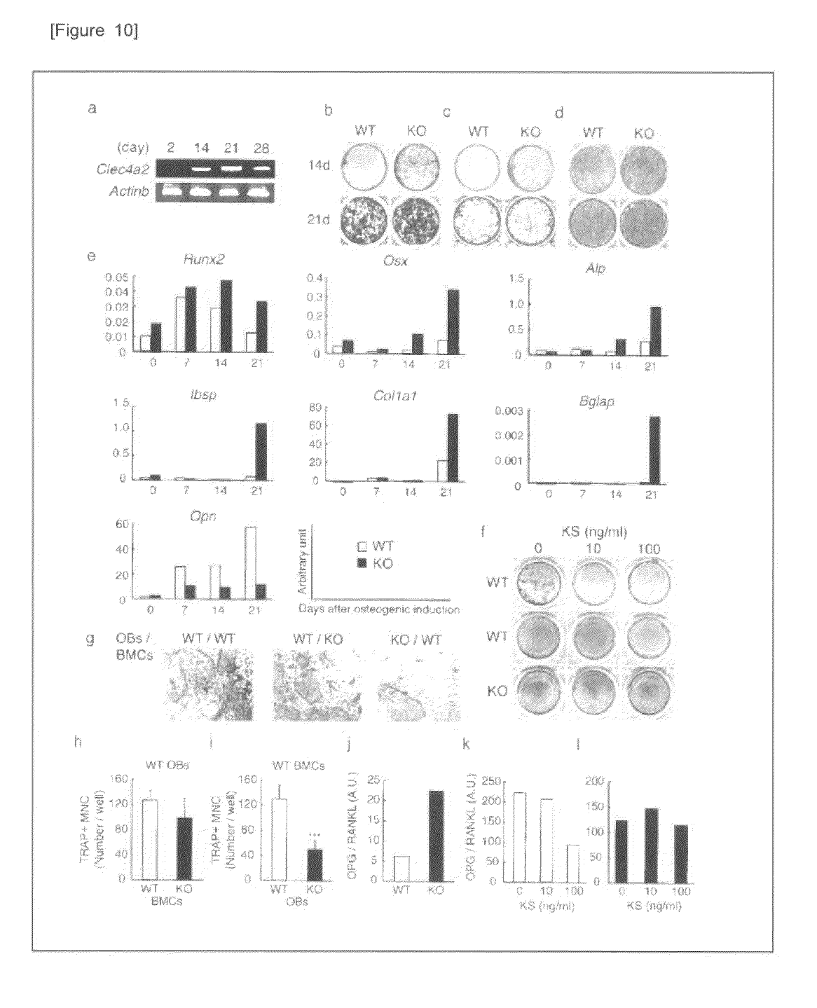

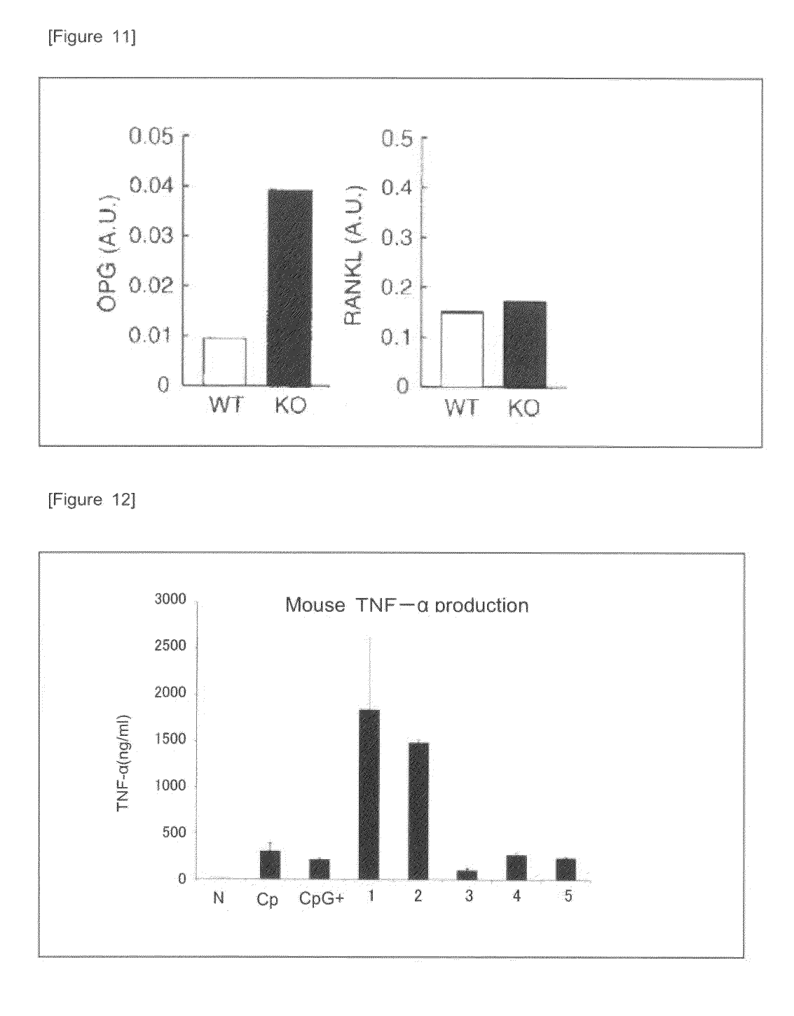

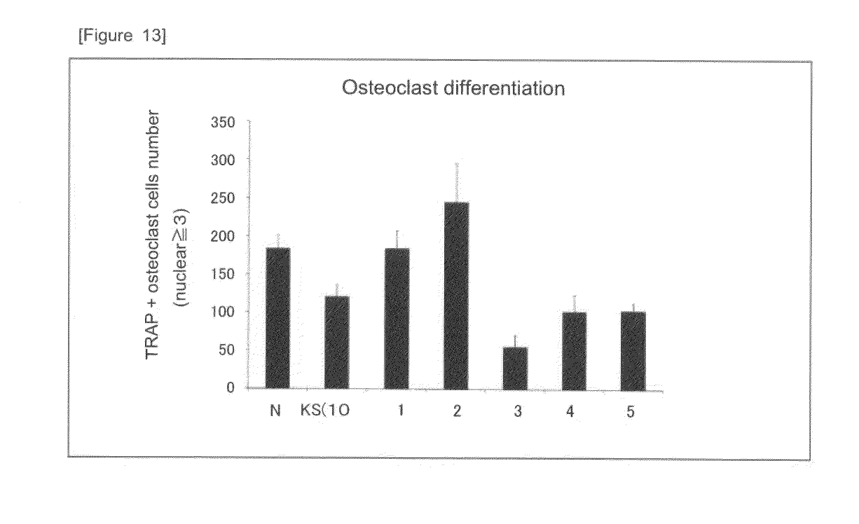

### BRIEF DESCRIPTION OF DRAWINGS

[FIG. 1] FIG. 1a is a schematic diagram illustrating structures of a mouse Dcir site (wild-type allele), a Dcir targeting construct (targeting vector), and a predicted mutant Dcir gene (mutant allele) in production of Dcir^{-/-} mice, wherein exons are shown by black boxes, Neo: neomycin-resistant gene, DT: diphtheria toxin gene, B: BamHI site, and E: EcoRI site, and black boxes having notation of 5′ probe and 3′ probe in the diagram respectively represent binding sites of 5′ probe and 3′ probe in Southern hybridization; FIGS. 1b to 1d show the results of Southern blot hybridization analysis of ES clones, wherein FIG. 1b: analysis using BamHI-cut genomic DNA and a 5′ probe, FIG. 1c: analysis using EcoRI-cut genomic DNA and a 3′ probe, and FIG. 1d: analysis using EcoRI-cut genomic DNA and a Neo probe; FIG. 1e is a diagram showing the results of genomic Southern blot analysis for confirming mouse Dcir deficiency; FIG. 1f is a diagram showing the results of Northern blot analysis of expression of Dcir mRNA in mouse spleen, wherein +/+: wild-type mouse, +/−: Dcir^{+/-}mouse, and −/−: Dcir^{-/-} mouse.

[FIG. 2] FIG. 2a shows three-dimensional micro-CT images (upper images) and transverse cross sections (lower images) of thighbones of 8-week old WT mice and KO mice; FIG. 2b are graphs showing, left-upper: ratio of bone volume to tissue volume (BV/TV), right-upper: trabecular number (Tb. N), left-lower: trabecular separation (Tb. Sp), and right-lower: trabecular spacing t (Tb. Spac), wherein data is shown as mean±s.d. (n=4 or 5/group); FIG. 2c shows toluidine blue-stained sections of thighbones of 8-week old WT mice and KO mice; FIG. 2d is a graph showing quantitatively measured values of growth plate thickness; FIG. 2e shows TRAP-stained sections of tibiae of 8-week old WT mice and KO mice; FIG. 2f is graphs showing the number of OCs (N. Oc) and the ratio of OC surface to bone surface (Oc. S/BS); FIG. 2g shows the results of dynamic histomorphometry analysis of thighbones of 8-week old WT mice and KO mice; and FIG. 2h is graphs showing mineral apposition rate (MAR) and bone formation rate (trabecular surface) (BFR/BS), wherein data is shown as mean±s.d. (n=4 or 6/group), *P<0.05, **P<0.01, and scale bar=100 μm.

[FIG. 3] FIG. 3 shows the results of micro-CT analysis of normal old KO mice, where FIG. 3a shows transverse cross section images of thighbones of WT mice and 12-month old KO mice without ankylosis; and FIG. 3b is a graph showing a ratio of bone volume to tissue volume (BV/TV), wherein data is shown as mean±s.d. (n=3/group).

[FIG. 4] FIGS. 4a and 4b show expression of Dcir (Clec4a2) in bones: the results of RT-PCR analysis of OC (bone marrow-derived osteoclast), OB (primary calvarial osteoblast), and chondrocyte (primary rib chondrocyte), where in the chondrocyte (FIG. 2b), since Dcir expression was not detected, Col2a1 and Col10a1 were measured as positive controls.

[FIG. 5] FIG. 5 shows bone development in KO mice, FIG. 5a is a growth curve on the basis of nasoanal length, wherein data is shown as mean±s.d. (n=8/group); and FIG. 5b shows alcian blue and alizarin red-stained bone images of newborn mice.

[FIG. 6] FIG. 6a is TRAP-stained images of bone marrow cell-derived OCs treated with M-CSF and RANKL; FIG. 6b shows the quantitatively measured numbers of TRAP-positive multinucleate cells (MNC: cell having at least three nuclei (left) or at least 20 nuclei (right)), wherein data is shown as mean±s.d. (n=6); FIG. 6c shows the results of resorption pit formation analysis, where OCs were cultured on a dentin slice; FIG. 6d shows quantitatively measured values of resorption pit area, wherein data is representative results of three or more separate experiments; FIG. 6e shows TRAP activity of OC culture supernatant, wherein data is shown as mean±s.d. (n=3); FIG. 6f shows the results of semi-quantitative RT-PCR analysis of main transcription factors (Nfatc1 and Nfatc2) that control OC differentiation and OC markers (Acp5 and Cask); FIG. 6g shows activities of MAPK (p38, ERK, and JNK), Akt, and NE-κB in bone marrow-derived macrophage (BMM) cell culture at various points of time after RANKL treatment; FIG. 6h shows the results of RANKL-induced tyrosine phosphorylation of PLCγ1 and PLCγ2 in the BMM cell culture; FIG. 6i shows TRAP-stained mononuclear cells after in vitro culture for two days; FIG. 6j shows the quantitatively measured numbers of TRAP-positive mononuclear cells, wherein data is shown as mean±s.d. (n=5 or 7/group); and FIG. 6k shows the results of proliferation assay using pOC after stimulation with M-CSF only or M-CSF and RANKL, wherein data is shown as mean±s.d. (n=3), *P<0.05.

[FIG. 7] FIG. 7a shows GM-CSF concentrations in culture media of culturing bone marrow cells containing M-CSF and RANKL, wherein data is shown as mean±s.d. (n=3); FIG. 7b shows the results of proliferation assay using pOC after stimulation with M-CSF only, M-CSF and RANKL, or M-CSF and GM-CSF, wherein data is shown as mean±s.d. (n=3 or 7/group); FIG. 7c shows influence of GM-CSF on osteoclast formation when a recombinant mouse GM-CSF in a concentration shown on the horizontal axis was added to a culture medium containing M-CSF (10 ng/mL) and RANKL (100 ng/mL), wherein data is shown as mean±s.d. (n=3); FIG. 7d shows influence of anti-GM-CSF neutralizing antibodies (Abs) on osteoclast formation when anti-GM-CSF antibodies (5 μg/mL) were added to a culture medium containing M-CSF (10 ng/mL) and RANKL (100 ng/mL), wherein data is shown as mean±s.d. (n=4); and FIG. 7e shows GM-CSF mediating phosphorylation of Stat5 in pOC, where whole-cell extracts were stimulated with GM-CSF and were collected at points of time shown in the drawing, and the concentration of each band was determined by densitometry and shown on the upper side of the Western blot image, wherein *P<0.05 and ***P<0.001.

[FIG. 8] FIG. 8a shows a structure of DCIR-binding carbohydrate, wherein Gal: galactose, GlcNAc: N-acetyl-D-glucosamine, and S: sulfate group; FIG. 8b shows concentration-dependent binding of recombinant mouse DCIR (mDCIR) and KS-II (mean±s.d.); FIG. 8c shows TRAP-stained OCs in the absence of KS-II (KS⁻) and in the presence of KS-II (KS⁺); FIG. 8d shows the number (mean±s.d.) of TRAP-positive MNCs (cells each containing at least three nuclei) in the absence and presence of KS-II (100 ng/mL); FIG. 8e shows osteoclast formation in the presence of carbohydrate chain (10 ng/mL), wherein KS-I: cornea-derived KS-I, KS-II: cartilage-derived KS-II, CS: chondroitin sulfate, DS: dermatan sulfate, and LacNAc: unsulfated LacNAc, wherein data is shown as mean±s.d.; and FIG. 8f shows the results of immunoblot analysis for ITIM phosphorylation and SHP-1 recruitment in pOC lysate after KS-II stimulation, where the complex was immunoprecipitated with an anti-DCIR antibody, and *P<0.05 and **P<0.01.

[FIG. 9] FIG. 9 shows the results of proliferation assay of new born mouse-derived primary calvarial OBs, where the primary OBs were isolated from the calvarial bone of the new born mouse and were cultured without inducing osteogenesis, wherein data is shown as mean±s.d. (n=5/group).

[FIG. 10] FIG. 10a shows the results of RT-PCR analysis of Dcir (Clec4a2) expression in OBs at a plurality of points of time after osteogenic differentiation; FIGS. 10b to 10d show mineralization of calvarial OBs, where osteogenic cultures were stained with alizarin red staining (FIG. 10b), von Kossa staining (FIG. 10c), and ALP staining (FIG. 10d); FIG. 10e shows the results of real-time RT-PCR analysis of OB marker mRNA expression in osteogenic cultures (21 days) of calvarial OBs, where Runx2: Runt-related gene 2 (Cbfa1: Core binding factor 1), Osx: Osterix (Sp7), Alp: Alkaline phosphatase, Ibsp: Integrin-binding sialoprotein (BSP: bone sialoprotein), ColIα1: Collagen type I alpha1, Osc: Osteocalcin, and Opn: Osteopontin; FIG. 10f shows influence of KS-II on calvarial OB mineralization, alizarin red staining (the upper) and ALP staining (the middle and the lower); FIG. 10g shows osteoclast formation in coculture containing Dcir^{-/-} mouse-derived BMCs and OBs; FIGS. 10h and 10i show the numbers of TRAP-positive MNCs after coculture of OBs and BMCs, wherein data is shown as mean±s.d. (n=4 to 11/group) and ***P<0.001; FIG. 10j shows the results of real-time RT-PCR analysis for the OPG/RANKL ratio in calvarial OBs; and FIGS. 10k and 10l show the results of real-time RT-PCR for influence of KS-II on the OPG/RANKL ratio in calvarial OBs derived from WT mice (FIG. 10k) and KO mice (FIG. 10l).

[FIG. 11] FIG. 11 shows the results of real-time RT-PCR analysis for expression levels of OPG and RANKL in calvarial OBs.

[FIG. 12] FIG. 12 shows influence of a hybridoma supernatant according to the present invention on TNF-α.

[FIG. 13] FIG. 13 shows influence of a hybridoma supernatant according to the present invention on osteoclast differentiation.

### EMBODIMENTS FOR CARRYING OUT THE INVENTION

The active ingredient of the DCIR agonist of the present invention is KS-II. Keratan sulfate is sulfated glycosaminoglycan having a basic structure of Gal-GlcNAc where N-acetylglucosamine (GlcNAc) binds to galactose (Gal). Keratan sulfate includes KS-II, which is derived from bone or cartilage and binds to protein by an O-glycoside bond, and KS-I, which is derived from cornea and binds to protein by an N-glycoside bond. In the present invention, only KS-II can be used, and KS-I does not have the activity of the present invention. Though KS-II is known to be involved in, for example, inflammatory (Patent Literature 1), involvement with DCIR was not known at all.

KS-II can have a variety of structures based on the repeat number of Gal-GlcNAc and the number of sulfate residues, and the KS-II of the present invention may have any structure.

The KS-II may be derived from bone or cartilage or may be of commercially available.

As shown in Examples described below, KS-II is a ligand for DCIR and exhibits various activities through binding to DCIR. That is, binding of KS-II to DCIR results in inhibition of maturation of osteoblasts and formation of a bone matrix, and promotion of production of osteopontin. Meanwhile, it was also revealed that binding of KS-II to DCIR results in strong suppression of formation of osteoclasts. In addition, the activity is caused by inhibiting proliferation of GM-CSF-dependent osteoclast precursor cells. KS-I is not a ligand for DCIR.

KS-II is therefore useful as an osteogenesis regulator and is useful as an agent for preventing or treating various bone diseases such as osteoporosis, Paget's disease of bone, and osteitis deformans.

Furthermore, since KS-II is a ligand for DCIR, it is possible to screen for a DCIR agonist or a DCIR antagonist by measuring binding affinity of a test substance to DCIR in the presence of KS-II or using KS-II as a control. More specifically, a test substance can be judged whether it is an agonist or an antagonist of DCIR by measuring binding affinities of KS-II and the test substance to DCIR and comparing the binding affinity of the test substance to DCIR with that of KS-II to DCIR. Here, the binding affinity to DCIR may be judged by measuring the activity of KS-II, e.g., osteoclast forming ability, activity on TNF-α production, or activity on type I IFN production.

The screening for a DCIR agonist or a DCIR antagonist can be performed either in vitro or in vivo. In the case of in vivo, osteoclast forming ability, activity on TNF-α production, or activity on type I IFN production is preferably measured for judgment.

The anti-DCIR antibodies of the present invention include anti-DCIR antibodies having a KS-II-like DCIR agonism (anti-DCIR agonistic antibodies) and anti-DCIR antibodies having a KS-II inhibitory DCIR antagonism (anti-DCIR antagonistic antibodies).

In the present invention, the antibody having a KS-II-like DCIR agonism may be one that has the DCIR agonism as in KS-II and includes one that causes the DCIR agonism through another material.

The antibody having a KS-II-like DCIR agonism preferably shows a binding affinity to DCIR equal to or higher than that of KS-II, with the binding affinity being preferably 1.2-times or more, more preferably twice or more, and even more preferably 5 times or more higher than that of KS-II. The binding affinity may be determined by directly measuring the binding affinity to DCIR or may be determined by using an indicative activity such as an osteoclast formation-suppressing activity or a TNF-α production-suppressing activity.

The antibody having a KS-II-like DCIR agonism in the present invention has an osteoclast formation-suppressing activity and a TNF-α production-inhibiting activity that are equal to or higher than those of KS-II. Accordingly, the KS-II-like anti-DCIR antibody is useful as an agent for preventing or treating a disease accompanied by abnormal bone metabolism, such as a bone resorption disease, an inflammatory disease or the like. Examples of the disease accompanied by abnormal bone metabolism and the inflammatory disease include osteoporosis, Paget's disease of bone, osteitis deformans, and rheumatoid arthritis. The effectiveness of the KS-II-like anti-DCIR antibody for these diseases can be confirmed, for example, using a collagen-induced arthritis model.

The antibody having a KS-II inhibitory DCIR antagonism preferably shows a binding affinity to DCIR equal to or higher than that of KS-II, with the binding affinity being preferably 1.2-times or more, more preferably twice or more, and even more preferably 5 times or more higher than that of KS-II.

The antibody having a KS-II inhibitory DCIR antagonism has a strong osteoclast formation-promoting activity and a TNF-α production-promoting activity. Accordingly, the KS-II inhibitory anti-DCIR antibody is useful as, for example, an anticancer agent, an immunostimulant or the like.

The anti-DCIR antibodies of the present invention include monoclonal antibody and polyclonal antibody as well as antibody mutants and derivatives such as antibodies maintaining ability of specifically binding to an epitope and T-cell receptor fragments.

Furthermore, the type of the antibody of the present invention is not particularly limited, and mouse antibodies, human antibodies, rat antibodies, rabbit antibodies, sheep antibodies, camel antibodies, a bird antibodies and the like as well as recombinant antibodies that have been artificially modified for, for example, reducing xenoantigenicity to human, such as chimeric antibodies and humanized antibodies. The recombinant antibody can be produced by a known method. The chimeric antibody consists of heavy and light chain variable regions of an antibody of a mammal other than human, such as a mouse, and heavy and light chain constant region of a human antibody and can be produced by linking a DNA encoding the variable region of a mouse antibody to a DNA encoding the constant region of a human antibody, inserting the resulting DNA into an expression vector, and introducing the construct into a host. The humanized antibody is also called a reshaped human antibody and is prepared by transplanting the complementarity determining region (CDR) of an antibody of a mammal other than human, for example, a mouse antibody into the complementarity determining region of a human antibody. A general gene recombination process for transplantation is also known. Specifically, a DNA sequence designed so as to link the CDR of a mouse antibody and the framework region (FR) of a human antibody to each other is synthesized by PCR from several oligonucleotides produced so as to have overlapping portions at the ends. The resulting DNA is linked to a DNA encoding a human antibody constant region, inserting it into an expression vector, and introducing the construct into a host, to thereby produce a humanized antibody (see European Patent Publication No. EP239400 and International Publication No. WO96/02576). The FR of a human antibody linked via the CDR is selected from FRs having the complementarity determining region which forms an appropriate antigen-binding site. If needed, an amino acid in a framework region of the variable region of an antibody may be replaced such that the complementarity determining region of a reshaped human antibody forms an appropriate antigen-binding site (Sato, K. et al., Cancer Res., 1993, 53, 851-856).

Furthermore, a method of preparing a human antibody is also known. For example, a desired human antibody having an antigen-binding activity can be prepared by sensitizing human lymphocytes in vitro with a desired antigen or cells expressing a desired antigen and fusing the sensitized lymphocytes with human myeloma cells such as U266 (see JP-B-1-59878). Furthermore, a desired human antibody can be prepared by immunizing a transgenic animal having all repertoires of human antibody genes with a desired antigen (see WO93/12227, WO92/03918, WO94/02602, WO94/25585, WO96/34096, and WO96/33735). Furthermore, a technology for producing a human antibody is obtained by panning using a human antibody library is also known. For example, the variable region of a human antibody is expressed as a single chain antibody (scFv) on the surface of a phage by a phage display method. A phage that binds to an antigen can be selected. A DNA sequence encoding the variable region of a human antibody that binds to the antigen can be determined by analyzing the gene of the thus selected phage. When the DNA sequence of scFv that binds to an antigen is revealed, an appropriate expression vector is constructed based on the sequence, and then a human antibody can be obtained. These methods are already known. Concerning these methods, WO92/01047, WO92/20791, WO93/06213, WO93/11236, WO93/19172, WO95/01438, and WO95/15388 can be referred to.

Furthermore, these antibodies may be lower molecular weight antibodies such as antibody fragments or modified products of the antibodies, as long as the specific characters are not lost. Specific examples of the antibody fragment include Fab, Fab′, F(ab′)2, Fv, and a diabody. Such an antibody fragment can be obtained by constructing a gene encoding the antibody fragment, introducing the gene into an expression vector, and then expressing the gene in an appropriate host cell (e.g., see Co, M. S. et al., J. Immunol., (1994) 152, 2968-2976; Better, M. and Horwitz, A. H., Methods Enzymol., (1989) 178, 476-496; Pluckthun, A. and Skerra, A., Methods Enzymol., (1989) 178, 497-515; Lamoyi, E., Methods Enzymol., (1986) 121, 652-663; Rousseaux, J. et al., Methods Enzymol., (1986) 121, 663-669; and Bird, R. E. and Walker, B. W., Trends Biotechnol., (1991) 9, 132-137).

As a modified product of an antibody, antibodies to which various molecules such as polyethylene glycol (PEG) is bound can be used. Such a modified product of an antibody can be obtained by chemically modifying the obtained antibody. Methods for modifying antibodies have already been established in the art.

The antibody and the antibody fragment of the present invention can be produced by any appropriate method, such as in vivo, cultured cells, in vitro translation reaction, or recombinant DNA expression system.

The methods of producing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands, 1984; St. Groth et al., J. Immunol. Methods, 35: 1-21, 1980). Any animal (e.g., mouse or rabbit) that is known to produce antibodies can be immunized through subcutaneous or intraperitoneal injection using DCIR or its fragment as an immunogen. In the immunization, an adjuvant may be used. Such an adjuvant is well known in the art.

Polyclonal antibodies can be obtained by isolating an antiserum containing antibodies from an immunized animal and screening for an antibody having a desired specificity by a method well known in the art, such as an ELISA, Western blot analysis, or radioimmunoassay.

Monoclonal antibodies can be obtained by removing spleen cells from an immunized animal and fusing the spleen cells with myeloma cells to produce hybridoma cells that produce monoclonal antibodies. A hybridoma cell that produces an antibody recognizing an intended protein or a fragment thereof is selected using a method well known in the art such as an ELISA, Western blot analysis, or radioimmunoassay. A hybridoma secreting the desired antibody is cloned and cultured under appropriate conditions. The secreted antibody is collected and purified by a method well known in the art, such as ion exchange column or affinity chromatography. Alternatively, a human monoclonal antibody may be produced by using a XenoMouse strain (see Green, J. Immunol. Methods, 231: 11-23, 1999; and Wells, Eek, Chem. Biol., 2000 Aug. 7(8): R185-6). Furthermore, at present, a monoclonal antibody is also produced using a phage display without immunization. The antibody of the present invention may be produced by any of these methods.

The pharmaceutical of the present invention can be formulated with a pharmaceutically acceptable carrier well known in the art by, for example, mixing, dissolving, granulating, tableting, emulsifying, encapsulating, or lyophilizing.

For oral administration, KS-II or the anti-DCIR antibody can be formulated with a pharmaceutically acceptable solvent, excipient, binder, stabilizer, dispersant or the like, into a dosage form such as a tablet, pill, sugar-coated pill, soft capsule, hard capsule, solution, suspension, emulsion, gel, syrup, or slurry.

For parenteral administration, KS-II or the anti-DCIR antibody can be formulated with a pharmaceutically acceptable solvent, excipient, binder, stabilizer, dispersant or the like, into a dosage form such as an injectable solution, suspension, emulsion, cream, ointment, inhalant, or suppository. For injectable formulation, the therapeutic agent of the present invention can be dissolved in an aqueous solution, preferably in a physiologically compatible buffer such as Hanks' solution, Ringer's solution, or a physiological saline buffer. Furthermore, the composition can take the form of, for example, a suspension, a solution, or an emulsion in an oleaginous or aqueous vehicle. Alternatively, KS-II or the anti-DCIR antibody may be produced in the form of powder, and an aqueous solution or a suspension may be prepared with sterilized water or the like before use. For administration by inhalation, KS-II or the anti-DCIR antibody is powdered and formulated into a powder mixture with a suitable base such as lactose or starch. The suppository formulation can be produced by mixing KS-II or the anti-DCIR antibody with a common suppository base such as cocoa butter. Furthermore, the therapeutic agent of the present invention can be formulated as a sustained-release preparation by encapsulating it into a polymer matrix or the like.

The dose of KS-II or the anti-DCIR antibody varies depending on the symptoms of a patient, the administration route, and the body weight and age of the patient, but is preferably 1 μg to 500 mg per adult per day, for example.

### EXAMPLES

The present invention will now be described in detail by Examples.

### (Experimental Method)

All animal experiments were approved by the Committee for Animal Use of the Institute of Medical Science the University of Tokyo and were performed in accordance with the safety guidelines for animal experiments and the ethical guidelines for gene replication experiments.

### 1. Production of Dcir Knockout (KO) Mice

Dcir^{-/-} (KO) mice were produced by a usual gene targeting approach according to the procedure described in Nature Medicine, 2008, vol. 14, no. 2: 176-180.

A targeting vector having a 5′ end homologous region, a BamHI site, an EcoRI site, a neomycin-resistant gene (Neo), and a diphtheria toxin gene (DT) for negative selection at the 3′ end was produced. Using this vector, exons 1 and 2 of the Dcir gene of mouse-derived ES cells were replaced with Neo to delete the genomic sequence mostly encoding cytoplasmic domain containing immunoreceptor tyrosine-based inhibitory motif (ITIM) and transmembrane domain (FIG. 1a). The genes from the ES clone were treated with BamHI and EcoRI and were subsequently screened by Southern blot hybridization analysis using a 5′ probe (FIG. 1b), a 3′ probe (FIG. 1c), and a Neo probe (FIG. 1d) to confirm gene deficiency. Dcir^{+/-} mice were produced using a Dcir-deficient ES clone, and Dcir^{-/-} mice were produced by mating the Dcir^{+/-} mice. Dcir^{-/-} mice were backcrossed with C57BL/6J (SLC) for eight to nine generations before they were used in experiments.

Deficiency of the Dcir gene in a mouse was confirmed by genomic Southern blot analysis (FIG. 1e). Deficiency of Dcir mRNA expression in spleen cells was confirmed by Northern blot hybridization analysis (FIG. 1f).

### 2. Analysis of Bone Phenotype

Mouse bone phenotype was analyzed by the following processes.

### (1) Computed Tomography (CT)

Three-dimensional micro-CT analysis of femora and joints was performed using R_mCT (manufactured by Rigaku Mechatoronics Co., Ltd.). Two-dimensional micro-CT analysis and quantitative determination of femora were performed using Scan Xmate-A0905 (manufactured by Comscantecno Co., Ltd.) and TRI/2D-BON system (manufactured by Ratoc System Engineering Co., Ltd.). Furthermore, peripheral bone quantitative CT (PQCT) analysis and quantitative determination offemora were performed using XCT Research SA+ system (manufactured by Stratec Medizitechnik GmbH).

### (2) Histologic Examination

HE staining of femora was performed in accordance with the method described in Nature Medicine, 2008, vol. 14, no. 2: 176-180. Joints were stained with toluidine blue (TB) and von Kossa, and tibias were stained with TB and TRAP. Staining was performed by fixing a sample with 10% neutral buffered formalin, treating the sample with a glycol methacrylate polymer or paraffin without demineralization, and slicing the sample into sections having a thickness of 3 μm. The growth plate at the proximal end of the tibia and trabecula were histomorphologically analyzed using Osteoplan II (manufactured by Carl Zeiss AG). Mice were subcutaneously injected with calcein (1.6 mg/kg of body weight) twice with an interval of three days for dynamic histomorphometry analysis. Four days after the first injection, the tibias were fragmented and were fixed with 70% ethanol. Unmineralized frozen sections having a thickness of 5 μm were produced using Leica CM3050S cryostat (manufactured by Leica Microsystems GmbH). Bone mineral apposition rate for bone and bone formation rate were analyzed using Zeiss Axioskop and Osteoplan II (manufactured by Carl Zeiss AG).

### 3. Analysis of Bone Development

The nasoanal length of each mouse was measured twice a week to draw a growth curve. A new born mouse was fixed with 100% ethanol for four days and was transferred in an acetone solution. Three days after, the mouse was washed with water and then stained with a staining solution composed of 1 part by weight of 0.1% alizarin red S (manufactured by Sigma Corp.)/95% ethanol, 1 part by weight of 0.3% alcian blue 6GX (manufactured by Sigma Corp.)/70% ethanol, 1 part by weight of 100% acetic acid, and 17 parts by weight of ethanol for 10 days. After washing with 96% ethanol, the specimen was stored in 20% glycerol/1% potassium hydroxide at room temperature until the osteogenesis is clearly recognized by sight, then transferred to 100% glycerol and stored therein.

### 4. In Vitro Osteoclast Formation and Pit Formation Assay

Nonadherent bone marrow cells were seeded in a well plate (in a 24-well plate at 5×10⁵ cells/well or in a E-well plate at 3×10⁶ cells/well) and were cultured in α-MEM (Gibco BRL) containing 10% FCS (manufactured by Biowest AG) and 10 ng/mL M-CSF (manufactured by R&D Systems, Inc.). After two days, nonadherent cells (including lymphocytes) were washed away, and remaining adherent cells were used as bone marrow-derived macrophages (BMMs). These osteoclast precursor cells (pOCs) were further cultured in the presence of 100 ng/mL of soluble RANKL (manufactured by PeproTech Inc. or Oriental Yeast Co., Ltd.) and 10 ng/mL of M-CSF to obtain osteoclasts. After three days, the osteoclasts were fixed in 10% neutral buffered formalin for three minutes, further kept in a mixture of ethanol/acetone (50:50 v:v) for 1 minute and subsequently incubated in a TRAP staining solution (Naphthol AS-MX phosphate: 5 mg, N,N-dimethylformamide: 0.5 mL, fast red violet LB salt: 30 mg, 0.1 M sodium acetate buffer (pH 5.0) containing 50 mM sodium tartrate: 50 mL) at room temperature. The number of TRAP-positive multinucleate cells (MNCs having three or more nuclei) was counted.

In the pit formation assay, osteoclasts were produced in the presence of 30 ng/mL M-CSF for two days and were subsequently treated with 30 ng/mL M-CSF and 150 ng/mL RANKL for further three days. The cells were then collected with trypsin, and the collected cells were seeded (in a 96-well plate at 1×10⁵ cells/well) and cultured on dentin slice using 30 ng/mL M-CSF and 150 ng/mL RANKL for two days. The sample was sonicated in 1 M NH₄OH and was stained with hematoxylin. The TRAP-negative MNCs and regions eroded by resorption pit were observed and measured with Biorevo BZ-9000 (manufactured by Keyence Corp.).

In order to investigate the influence of GM-CSF on osteoclast formation, mouse GM-CSF enzyme immunoassay (manufactured by PerSeptive Biosystems, Inc.), recombinant GM-CSF (manufactured by PeproTech Inc.), and anti-mouse GM-CSF neutralizing antibody (manufactured by R&D Systems, Inc.) were used. Furthermore, in the proliferation assay, pOCs were seeded (in a 96-well plate at 1×10⁴ cells/well) and cultured in the presence of 10 ng/mL M-CSF only or M-CSF and 100 ng/mL RANKL or 20 ng/mL GM-CSF for three days. Subsequently, the cells were exposed to [³H]TdR (0.5 μCi/mL) overnight.

### 5. Detection of Differentiation and Mineral Apposition of Osteoblast

### (1) Culture of Primary Osteoblasts

Primary osteoblasts were isolated from the calvaria of a new born mouse. The calvaria was washed with PBS and was digested in α-MEM containing 0.25% trypsin and 2 mg/mL collagenase P (manufactured by Roche) at 37° C. for 20 minutes. The supernatant was removed, and the cells were further digested for 60 minutes.

In order to induce osteogenesis, primary osteoblasts (in a 96-well plate at 2×10⁴ cells/well, in a 24-well plate at 5×10⁴ cells/well, or in a 6-well plate at 2×10⁵ cells/well) were cultured to confluent, and the culture medium was replaced by a culture medium containing 10 mM β-glycerophosphate and 50 μg/mL ascorbic acid. The culture medium was replaced three times in 21 days.

The osteoblasts cultured for 21 days were stained with alizarin red, von Kossa, and alkaline phosphatase (ALP).

### (2) Alizarin Red Staining

The culture solution was removed from the osteoblasts cultured for 21 days by using PBS, and the osteoblasts were fixed with 3.7% formalin solution (Nacalai Tesque, Inc.) for 10 minutes. Subsequently, the cells were washed with PBS once again and were reacted with an ALP staining solution (0.1 mg/mL Naphtol AS-MX phosphate (Nacalai Tesque, Inc.), 0.6 mg/mL Azoic Diazo Component 20 (TGI), 5 μL/mL N,N-dimethylformamide (Nacalai Tesque, Inc.), and 7.5 mL Tris-HCl (1.5 M, pH 8.8) (Nacalai Tesque, Inc.)) for 20 minutes. The reaction solution was removed, and the cells were washed with PBS and then dried, followed by photographing with BIOREVO (KEYENCE Corp.).

### (3) Von Kossa Staining

The culture solution was removed from the osteoblasts cultured for 21 days by using ion-exchange water, and the osteoblasts were fixed with 3.7% formalin solution for 30 minutes. Subsequently, the cells were washed with ion-exchange water once again and were reacted with 5% silver nitrate solution (Nacalai Tesque, Inc.) under direct sunlight for 15 minutes. In order to terminate the reaction, the 5% silver nitrate solution was removed. To the residue, 5% sodium thiosulfate (Nacalai Tesque, Inc.) was added, and the mixture was left to stand for 2 minutes. The 5% sodium thiosulfate was washed with ion-exchange water and then dried, followed by photographing with BIOREVO (KEYENCE Corp.).

### (4) ALP Staining

Primary osteoblasts were cultured in a 96-well plate for 21 days and were measured with a TRACP & ALP Assay Kit (TaKaRa Bio Inc.). The cell culture supernatant was collected, and the undiluted solution and 10-fold diluted solution were used as templates. A reaction substrate solution was added to 50 W., of a template for a reaction at 37° C. for 15 minutes. The reaction was terminated with 50 μL of a reaction terminating solution, and subsequently the activity was measured with MICROPLATE READER MTP-300 (CORONA ELECTRIC Co., Ltd.) at OD405.

### 6. Co-Culture of Primary Osteoblast and Bone Marrow Cell (BMC)

Primary osteoblasts and BMCs (at 3×10³ cells/well and 2×10⁵ cells/well, respectively, in a 48-well plate) were cultured in 10⁻³ M 1.25(OH)₂D₃ (manufactured by Sigma Corp.) and 10⁻⁷ M PGE₂ (manufactured by Nacalai Tesque Inc.) for eight days. The culture medium was replaced every two days. TRAP staining was performed, and the number of MNCs was counted.

### 7. RT-PCR Analysis

Semi-quantitative RT-PCR and real-time RT-PCR were slightly modified, but basically performed in accordance with the methods described in Nature Medicine, 2008, vol. 14, no. 2: 176-180 and Arthritis Res. Ther., 2006, 8, R100, 1-13. Total RNA was prepared by a typical acid guanidinium thiocyanate-phenol-chloroform method or a method using Sepasol-RNA I Super (manufactured by Nacalai Tesque, Inc.). The prepared RNA was reverse transcribed with SuperScript III First-Strand Synthesis System for RT-PCR (manufactured by Invitrogen) to prepare cDNA.

The semi-quantitative RT-PCR was performed using the cDNA prepared above as a template. A reaction solution for each sample was prepared to be 20 μl and to contain 2 μl of 10×PCR reaction buffer (Roche), 1.6 μl of dNTPs (Roche), 0.4 μl of each primer shown in Table 1, 0.2 of Taq DNA polymerase (Roche), and 1 μl of template DNA. After that, amplification reaction was performed using iCycler (Bio-Rad Laboratories, Inc.). The reaction solution after the PCR was subjected to electrophoresis by using a 1.5% agarose gel at a constant voltage of 100 V for 30 minutes to separate the PCR products, which were stained with ethidium bromide (0.05 μg/mL) for 15 minutes to detect DNA fragments using an UV illuminator (BAS-III).

The real-time RT-PCR was performed using primers shown in Table 1, SYBR Green qPCR kit (manufactured by Invitrogen), and iCycler (manufactured by Bio-Rad Laboratories, Inc.).

TABLE 1
Primer sequence

| | | |
|---|---|---|
| collagenlal | Forward; 5′-GGTGCCCCCGGTCTTCAG-3′ | (SEQ ID NO: 1) |
| | Reverse; 5′-AGGGCCAGGGGGTCCAGCATTTC-3′ | (SEQ ID NO: 2) |
| Osteocalcin | Forward; 5′-CTGACCTCACAGATCCCAAGC-3′ | (SEQ ID NO: 3) |
| | Reverse; 5′-TGGTCTGATAGCTCGTCACAAG-3′ | (SEQ ID NO: 4) |
| Osteopontin | Forward; 5′-TAGCTTGGCTTATGGACTGAGG-3′ | (SEQ ID NO: 5) |
| | Reverse; 5′-AGACTCACCGCTCTTCATGTG-3′ | (SEQ ID NO: 6) |
| bone siaro protein | Forward; 5′-ACAATCCGTGCCACTCACT-3′ | (SEQ ID NO: 7) |
| | Reverse; 5′-TTTCATCGAGAAAGCACAGG-3′ | (SEQ ID NO: 8) |
| ALP | Forward; 5′-GGACAGGACACACACACACA-3′ | (SEQ ID NO: 9) |
| | Reverse; 5′-CAAACAGGAGAGCCACTTCA-3′ | (SEQ ID NO: 10) |
| RunX2 | Forward; 5′-TGTTCTCTGATCGCCTCAGTG-3′ | (SEQ ID NO: 11) |
| | Reverse; 5′-CCTGGGATCTGTAATCTGACTCT-3′ | (SEQ ID NO: 12) |
| Osterix | Forward; 5′-CCCACCCTTCCCTCACTCAT-3′ | (SEQ ID NO: 13) |
| | Reverse; 5′-CCTTGTACCAGCCATAGG-3′ | (SEQ ID NO: 14) |
| RANKL | Forward; 5′-CAGCATcGCTCTGTTCCTGTA-3′ | (SEQ ID NO: 15) |
| | Reverse; 5′-CTGCGTTTTCATGGAGTCTCA-3′ | (SEQ ID NO: 16) |
| OPG | Forward; 5′-ACCCAGAAACTGGTCATCAGC-3′ | (SEQ ID NO: 17) |
| | Reverse; 5′-CTGCAATACACACACTCATCACT-3′ | (SEQ ID NO: 18) |
| GAPDH | Forward; 5′-TTCACCACCATGGAGAAGGC-3′ | (SEQ ID NO: 19) |
| | Reverse; 5′-GGCATGGACTGTGGTCATGA-3′ | (SEQ ID NO: 20) |

### 8. Immunoblot

### (1) Western Blot

Western blot was performed in accordance with the method described in Nature Medicine, 2008, vol. 14, no. 2: 176-180. As pretreatment, a PVDF membrane (Bio-Rad Laboratories, Inc.) was impregnated with methanol and was then moved into an electrotransfer buffer (25 mM Tris-HCl (pH 8) (Nacalai Tesque, Inc.), 15 mg/mL Glycine (Nacalai Tesque, Inc.), and 20% methanol (Wako) for about 30 minutes for permeation. Transfer to the PVDF membrane was performed using TRANS-BLOTSD SEMI-DRY TRANSFER CELL (Bio-Rad Laboratories, Inc.) at a current of 2 mA per cm² of gel area for 1 hour. After the transfer, the PVDF membrane was blotted with a primary antibody, which is an antibody specific to any of the following proteins:

Phospho-p38 MAPK (Thr180/Tyr182);

p38 MAPK;

Phospho-p44/42 MAPK (Thr202/Tyr204);

p44/42 MAPK (137F5);

Phospho-SAPK/JNK (Thr183/Tyr185:81 E11);

SAPK/JNK (56G8);

Phospho-Akt (Thr308, C31E5);

Akt (pan) (C67E7);

Phospho-NF-κB p65 (Ser536, 93H1);

NF-κB p65 (C22B4);

Phospho-PLCγ1 (Tyr783);

PLCγ1;

Phospho-PLCγ2 (Tyr759);

PLCγ2; (3-tubulin (Abcam);

Phospho-Stat5 (Tyr694) (Cell Signaling Technology); and

Stat5 (C17) (Santa Cruz Biotechnology).

As the secondary antibody, anti-rabbit IgG, HRP-Linked Antibody (Cell Signaling) was used. The membrane was washed, and luminescence using ECL-Plus (GE Healthcare) was analyzed with FLA-5000 (FUJIFILM Corp.).

### (2) Immunoprecipitation Analysis

In immunoprecipitation analysis, Protein G-Sepharose 4 Fast Flow (manufactured by GE Healthcare), mouse DCIR-specific antibody (320511: manufactured by R&D Systems, Inc.), phospho-Tyr (4G10: manufactured by Millipore), and SHP-1 (HG213: Upstate) were used. As the secondary antibody, a rabbit IgG-specific HRP-linked polyclonal antibody (manufactured by Cell Signaling) or a rat IgG-specific HRP-linked polyclonal antibody (manufactured by Zymed) was used.

### 9. Carbohydrate Chain and Carbohydrate Chain Binding Assay

A purified product of bovine articular cartilage-derived keratan sulfate (KS-II) was obtained from K. Yoshida (Riken) and A. Tawaza (Hydrox Inc.). Other carbohydrate chains (bovine cornea-derived keratan sulfate, whale joint-derived chondroitin sulfate A, porcine skin-derived chondroitin sulfate (dermatan sulfate), and N-acetyllactosamine) were purchased from Seikagaku Biobusiness Corporation.

Carbohydrate chain binding assay was performed in accordance with the method described in J. Bio. Chem., 2004, 279, 29043-29049. KS-II was diluted to 2 mg/mL with 100 mM sodium acetate (pH 5.5) and was oxidized with 2 mM sodium metaperiodate on ice for 1 hour to obtain a reactive aldehyde group. After further dilution, KS-II was incubated on a Covalink ELISA plate at 4° C. overnight and was reduced with 0.3% sodium cyanoborohydride at room temperature for 1.5 hours for covalent bonding. The plate was washed and was blocked with 2% bovine serum albumin (fraction V: manufactured by Sigma Corp.) at 37° C. for two hours. A recombinant mouse DCIR (mDCIR) containing an extracellular domain (99th to 238th residues) was expressed as an inclusion body in E. coli BL21 (DE3) pLysS using a pGMT7 plasmid and the blocked plate was refolded by a typical dilution method. The mDCIR was incubated on the blocked and refolded plate at 4° C. overnight. Binding of protein was detected using anti-DCIR antibodies (a mixture of 320507 and 320511: manufactured by R&D Systems, Inc.) and HRP-linked rat IgG (manufactured by Zymed) in the presence of a TMB substrate (manufactured by Dako).

### Example 1

### Osteogenesis in Dcir^{-/-} (KO) Mouse

Dcir^{-/-} (KO) mice have been reported to develop enthesitis-induced ankylosis with aging (Nature Medicine, 2008, vol. 14, no. 2: 176-180). Bone volumes of young (8-week old) KO mice that had not developed ankylosis and normal old (12-month old) KO mice that did not show ankylosis symptoms were measured by three-dimensional micro-CT and were compared with that of wild-type mice. The bone volumes of the young KO mice and normal old KO mice were higher than that of wild-type (WT) mice (FIGS. 2a, 2b, and 3). It was therefore shown that osteogenesis is promoted in Dcir^{-/-} mice showing moderate osteosclerosis.

Then, the expression of DCIR in the bone of mice was measured by PT-PCR. DCIR was expressed in bone marrow-derived osteoclasts (OCs) and primary osteoblasts (OBs), but was not expressed in primary chondrocytes (FIG. 4). This result also supports involvement of Dcir in bone metabolism. The growth curve based on nasoanal length and the results of bone staining of new born mice with alizarin blue and alizarin red show that bone normally develops even in Dcir^{-/-} mice (FIG. 5).

Osteosclerosis is observed in animals having disorder of chondrocyte, OC, or OB (Genes Dev, 1999, 13, 3037-3051). In order to investigate causes of osteosclerosis in Dcir^{-/-} mice, the bone of mice was examined by histomorphometry analysis. The thickness of growth layer at proximal site of the tibia growth plate of KO mice does not differ from that of WT mice. Thus, it was shown that chondrocytes are normally formed even in KO mice (FIGS. 2c and 2d).

The number of TRAP-positive OCs and the OC surface area in the trabecula were significantly increased in KO mice (FIGS. 2e and 2f). Considering the enhancement of osteogenesis of KO mice that do not show ankylosis development, this result is unexpected.

Furthermore, in order to investigate bone turnover in these mice, bone newly formed in 8-week old mice was measured by dynamic histomorphometry analysis. That is, calcein was administered to mice twice with an interval of three days to label the bone, and the bone formed during that time was measured. The formation rate of the tibia and mineralization in KO mice were higher than those in WT mice (FIGS. 2g and 2h). It was therefore shown that in Dcir^{-/-} mice, not only bone resorption by osteoclasts but also osteogenesis by osteoblasts are enhanced to accelerate bone turnover. Since the bone density is increased as a whole in Dcir^{-/-} mice, under physiological conditions, the effect on osteogenesis is higher than that on bone resorption.

### Example 2

### Role of DCIR in Osteoclast Formation

The above-described example shows that Dcir^{-/-} mice have a large number of osteoclasts (OCs). Accordingly, the role of DCIR in osteoclast formation was investigated. Primary bone marrow cells (BMCs) derived from WT and KO mice were cultured in a standard in vitro OC differentiation system using a macrophage colony stimulating factor (M-CSF) and a receptor activator of nuclear factor KB ligand (RANKL) to induce OC differentiation. As a result, in the Dcir^{-/-} BMC culture product, differentiation into TRAP-positive multinucleate OCs was significantly increased (FIGS. 6a and 6b). Many of KO mouse-derived OCs were multinucleate cells and were similar to the bone phenotype of bone Paget's disease patients. In the Dcir^{-/-} OC, the size of the pit formed by bone resorption was enlarged, which clarified that the bone resorption activity of the Dcir^{-/-} OC was highly enhanced (FIGS. 6c and 6d). In addition, in the culture media containing Dcir^{-/-} cells, the TRAP activity was increased (FIG. 6e). These results show that DCIR participates in differentiation of BMC to mature OC.

Then, in order to evaluate influence of DCIR on multinucleate OC formation, expression amounts of nuclear factor of activated T-cells 1 (Nfatc1) and nuclear factor of activated T-cells 2 (Nfatc2) in Dcir^{-/-} OC were investigated. Nfatc 1 and Nfatc 2 are both main regulators for OC maturity and are OC specific markers, like Acp5 (TRAP) and Ctsk (cathepsin K). Expression amounts of Nfatc 1 and Nfatc 2 genes were equivalent to that of wild-type OC (FIG. 6f). It was therefore shown that DCIR does not affect NFAT expression.

The OC differentiation from BMC is regulated by a signaling pathway that is activated by RANK and immunoreceptor tyrosine-based activation motif harboring adaptor (ITAM-containing adaptor) (Nature, 2004, 428, 758-763). Accordingly, signaling of RANK in these cells was investigated. Activation of MAPK (p38, ERK, JNK), Akt, and NF-κB (all are located downstream of TRAF6) for RANKL induction is normal, and there is no difference between WT OC and mature OC for RANKL-induced ITAM-containing adaptor-dependent tyrosine phosphorylation of PLCγ1 and PLCγ2 (FIGS. 6g and 6h). These data shows that DCIR does not affect the RANKL-dependent OC differentiation from OC precursor cell (pOC).

In order to observe influence of Dcir deficiency in the initial stage of OC differentiation, the number of TRAP-positive mononuclear OCs was counted on two days after the in vitro culture. The number of mononuclear OCs significantly increased in Dcir^{-/-} BMCs treated with M-CSF (FIGS. 6i and 6j), which shows that DCIR has any role in the initial stage of OC differentiation. On the other hand, there was no difference in proliferative response when WT and Dcir^{-/-} pOCs were treated with M-CSF and RANKL (FIG. 6k). This result shows that DCIR negatively regulates OC formation without inhibiting RANKL-dependent signaling, which is believed to be a main signaling cascade for osteoclast formation.

Furthermore, reaction of pOC against GM-CSF was investigated. Conventional reports show that GM-CSF promotes differentiation of bone marrow cell precursor to dendritic cell and therefore inhibits expression of OCs (Blood, 2001, 98, 2544-2554). Recently, however, it was reported that GM-CSF can promote differentiation, proliferation, existence, and fusion of pOCs under specific conditions (J Bone Miner Res, 2004, 19, 190-199; Nat Med, 2007, 13, 62-69; Nat Med, 2008, 14, 81-87; J Immunol, 2009, 183, 3390-3399; and Biochem Biophys Res Commun, 2008, 367, 881-887). Accordingly, the influence of GM-CSF on osteoclast formation was investigated using cultured cells.

GM-CSF significantly increased differentiation of Dcir^{-/-} pOCs, not of WT pOCs (FIG. 7a). There was no difference in concentration of GM-CSF between the culture product of WT mice and Dcir^{-/-} mice (FIG. 7b). When compared with WT BMC, OC differentiation from Dcir^{-/-} BMC significantly increased in the presence of 0.1 ng/mL of recombinant GM-CSF, but not increased in the presence of 1 ng/mL or more of recombinant GM-CSF (FIG. 7c). In contrast, osteoclast formation in Dcir^{-/-} mice was considerably suppressed by treatment with anti-GM-CSF neutralizing antibody (FIG. 7d). Phosphorylation of signaling factor and transcription factor 5 (Stat-5) activator in Dcir^{-/-} pOC after GM-CSF stimulation was investigated. GM-CSF-induced Stat-5 phosphorylation of Dcir^{-/-} cells was considerably up-regulated (FIG. 7e). These results show that DCIR regulates the pOC proliferation by inhibiting the GM-CSF signaling.

### Example 3

### Identification of DCIR Ligand

No ligand for DCIR has been reported until now. The present inventors have accordingly searched for a carbohydrate chain that binds to DCIR using a public glycan array database for identifying in vivo DCIR ligand participating in osteoclast formation. As a result, a carbohydrate chain having a structure of sulfated galactose-β-1-4-N-acetyl-D-glucosamine (N-acetyllactosamine or LacNAc) was identified as a DCIR ligand (FIG. 8a). Sulfated poly-LacNAc is a molecule existing inside keratan sulfate (KS), which is mainly present in cornea (N-binding type KS-I) and articular cartilage (O-binding type KS-II), as a sulfated glycosaminoglycan (GAG) side chain of proteoglycan. Accordingly, the inventors have investigated expression of KS in mouse joints and bone marrow cells (BMCs) to confirm that KS is expressed in these sites. Furthermore, the results of investigation of binding between DCIR and KS through carbohydrate chain binding assay by ELISA revealed that recombinant mouse DCIR concentration-dependently binds to KS-II derived from bovine articular cartilage (FIG. 8b).

Furthermore, influence of KS on OC differentiation was evaluated by counting the number of TRAP-positive OCs in vitro. KS-II considerably inhibited OC formation in wild-type (WT) bone marrow cell culture product, but did not inhibit in Dcir^{-/-} cell culture (FIGS. 8c and 8d). OC differentiation was not influenced by cornea-derived KS-I, unsulfated LacNAc, and other sulfated glycosaminoglycans such as chondroitin sulfate (CS) and dermatan sulfate (DS) (FIG. 8e). Furthermore, in order to confirm that DCIR is activated by KS, immunoprecipitation analysis was performed. As a result, it was revealed that KS-II concentration-dependently accelerates phosphorylation of immunoreceptor tyrosine-based inhibitory motif (ITIM) and recruitment of tyrosine phosphatase SHP-1 in DCIR (FIG. 8f). These results show that KS-II is a ligand specific to DCIR and participates in inhibition of osteoclast formation through activation of DCIR.

### Example 4

### Role of Dcir in Osteoblast (OB) Formation

Roles of Dcir in osteoblast (OB) formation were investigated. There was no difference in proliferation of primary parietal bone OBs between WT mice and Dcir^{-/-} mice (FIG. 9). In OBs derived from calvaria, Dcir was expressed after induction of osteogenesis differentiation by β-glycerophosphoric acid and ascorbic acid, and the expression continued all over during the mineralization process (FIG. 10a). In Dcir^{-/-} OBs, addition of calcium (FIG. 10b) and calcium phosphate (FIG. 10c) and expression of alkaline phosphatase (ALP) (FIG. 10d) were enhanced in the initial stage of culture (14 days). This revealed that mineralization is promoted in Dcir^{-/-} OBs compared with that in WT OBs. These results suggest that DCIR participates in end-OB differentiation and mineralization matrix formation.

The expression amounts of genes encoding OB markers such as Runx2, Osterix, and ALP and bone matrix proteins such as bone sialoprotein (BSP), type I collagen (ColI), and osteocalcin were high in Dcir^{-/-} mice (FIG. 10e). The expression amount of osteopontin (OPN) necessary for bone resorption was considerably low in osteoblasts of Dcir^{-/-} mice compared with that in osteoblasts of WT mice. These data suggest that DCIR inhibits maturation of osteoblasts and formation of bone matrix and promotes of OPN production, to thereby negatively regulate osteogenesis. Furthermore, alizarin red staining and ALP staining of osteogenic culture product revealed that presence of KS-II suppresses mineralization of WT OBs, but does not suppress mineralization of Dcir^{-/-} OBs.

Roles of osteoblast in regulation of osteoclast formation were investigated by co-culture of Dcir^{-/-} mouse-derived BMCs and OBs. The co-culture product of Dcir^{-/-} mouse-derived BMCs and WT mouse-derived OBs showed normal osteoclast formation (FIGS. 10g and 10h). In contrast, in co-culture product of WT mouse-derived BMCs and Dcir^{-/-} mouse-derived OBs, the osteoclast formation was highly decreased (FIG. 10g and 10i). This result shows that DCIR participates in normal coupling of osteoblast and osteoclast. Since the OC formation is regulated by osteoblast-derived RANKL and osteoprotegelin (OPG), their expression amounts in Dcir^{-/-} OBs were investigated. Though no substantial difference in RANKL expression amount was observed, the expression amount of OPG was significantly increased in Dcir^{-/-} OBs compared with that in WT mouse-derived OBs, and the OPG/RANKL ratio of mutant osteoblast was increased (FIGS. 10j and 11). Furthermore, KS-II treatment decreases the OPG/RANKL ratio of only WT mouse-derived OBs (FIGS. 10k and 10l). This shows that KS-II DCIR-dependently regulates OPG production and therefore suggests that DCIR promotes osteoclast formation by inhibiting OPG expression. In addition, it is believed that DCIR functions not only in osteoclast but also in osteoblast to regulate bone turnover in vivo.

### Example 5

### Preparation of Anti-DCIR Antibody

### (1) Preparation of DCIR-Expressing Cell

An expression vector containing a Dcir gene was prepared by amplifying 137th to 989th nucleotides of a sequence (NM_{—}011999) containing a Dcir gene by PCR and inserting it into a pcDNA3.1+ vector and was transduced into 293T cells and COST cells using Lipofectamine 2000 (Invitrogen). After about 48 hours, the cells were collected and cryopreserved.

### (2) Confirmation of DCIR Expression

Transient expression cells prepared in (1) were subjected to confirmation of DCIR expression by performing Western blot of DCIR-expressing cells and their wild-type cells using an anti-DCIR antibody and detecting a band at around 35 kDa, which is expected to be specific to DCIR-expressing cells. Thus, DCIR-expressing cells were obtained.

### (3) Immunization of Mouse by Foot Pad Method

Mice (four mice) were immunized with an emulsion of a mixture of an adjuvant (complete adjuvant (FREUND), RM606-1, Mitsubishi Chemical Iatron, Inc.) and PBS from the sole of one foot. After one week, the mice were immunized with the DCIR-expressing cells prepared in (2). The immunization was performed once a week, five times in total.

### (4) Cell Fusion

Three days after the fifth immunization, enlarged lymph nodes were extracted from both feet of the immunized mice, and cells were collected therefrom. Myeloma cells (P3U1) were multiplied in a culture flask (culture medium: 10% FBS-RPMI), and the cells were collected. The collected lymph node-derived cells were mixed with the myeloma cells, followed by centrifugation. To the resulting pellet, PEG (PEG 4000: MERCK Cat. No. 1097270100, diluted with the same amount of RPMI) was added for cell fusion. The cells were washed with a serum-free RPMI medium (RPMI1640, SIGMA Cat. No. R8758), were suspended in a 15% FBS-HAT medium (HAT supplement (50×): GIBCO Cat. No. 21060-017, addition of supplement for rescuing unstable hybridomas in the initial stage) and were seeded in four 96-well plates. The culture medium was replaced three days after the seeding. After confirmation of formation of hybridoma colonies (after two to three weeks), the culture supernatant was collected from the well plates and was subjected to primary screening.

### (5) Primary Screening for Hybridoma

Primary screening for hybridomas was performed by cell ELISA/flow cytometry (FCM).

Cell ELISA: The cryopreserved cells (transfectant) were initiated, suspended in 0.5% BSA/2 mM EDTA/PBS, and dispensed in a Cell ELISA plate (NUNC 249570 96V NW PS) at 100 μL/well, which corresponds to 1×10⁷ cells per one 96-well plate. After centrifugation at 2000 rpm for 2 minutes at 4° C., the supernatant was discarded, and the culture supernatant collected in (4) was added to the plate at 50 μL/well, followed by reaction at room temperature for 30 minutes. After washing with 0.5% BSA/2 mM EDTA/PBS (the supernatant centrifuged at 2000 rpm for 2 minutes at 4° C. was discarded) twice, a goat anti-mouse IgG-POD labeled antibody (MBL product Code 330) diluted by 10,000 times with a buffer (diluent manufactured by MBL Corp.) was added thereto, followed by reaction at room temperature for 30 minutes. After washing three times, a chromogenic substrate was added thereto. After color development for 5 to 10 minutes, absorbance was measured at 450 to 620 nm.

FCM: The reactivity of the culture supernatants of clones that may be Cell ELISA positive to the DCIR-expressing cells prepared in (2) was confirmed by FCM. Undiluted culture supernatant was reacted with the cells at room temperature for 30 minutes. After washing twice, an FITC-labelled anti-mouse IgG antibody (MBL product, IM-0819) diluted by 100 times was reacted for 30 minutes. After washing twice, the cells were suspended in 400 μL of buffer, followed by measurement with FC500 (Beckman Coulter Inc.). As the buffers for washing and suspension of the cells, 0.5 mM EDTA, 5% BSA, and PBS were used. In order to avoid artifacts such as false-positive, reactivity was confirmed several times during subculture.

### (6) Monoclonization

Hybridomas selected from culture supernatant determined to be positive by primary screening for hybridomas in (5) were monoclonized.

Hybridomas in the logarithmic growth phase were pipetted with a Pasteur pipette and were then collected. After dilution with the culture medium, the cell concentration was adjusted to 1 to 32000 cells per well. The cells were seeded in a 96-well plate. After confirmation of single colony formation of a hybridoma (after one to two weeks), the culture supernatant was collected from the well plate, and the activity thereof was confirmed in accordance with the method in (5).

### (7) Confirmation of Monoclonal Antibody (Isotype)

The culture supernatant diluted by 100 times with PBS was dropwisely added to a development tube to resuspend colored latex beads. A strip for isotyping (Iso Strip mouse monoclonal antibody isotyping kit: Roche, Cat. No. 1-493-027) was immersed in the tube. After 5 minutes, bands detected at specific subclasses were confirmed to select clones.

### (8) Freezing of Monoclonal Hybridoma

Monoclonal hybridomas were subcultured from one well of a 96-well plate, to a 48-well plate, 24-well plate, and 12-well plate. Cells in one well were collected by centrifugation, suspended in 500 μL of Cellbanker (Juji Field Inc., Cat. No. BLC-1), put in a stock tube (SUMILON, Cat. No. MS-4601W), and stored at −80° C. The culture supernatants collected during freezing were each prepared into 10 mL of a final evaluation medium. The final evaluation culture supernatant was used for final confirmation whether a desired activity is maintained or not.

### Example 6

### Evaluation of Anti-DCIR Antibody

The anti-DCIR antibodies derived from hybridomas prepared in Example 4 were evaluated for binding ability to DCIR. Since DCIR has been reported to participate in suppression of immunoreaction (Nature Medicine, 2008, vol. 14, no. 2: 176-180), the DCIR-binding ability of each antibody was evaluated using expression of tumor necrosis factor α (TNF-α), which is an inflammatory cytokine, as a standard.

The hybridoma prepared in Example 4 was cultured in a culture medium containing CpG (concentration: 1 μM) for 12 hours, and the culture supernatant was collected. Separately, CpG ODN 1668 (Operon Technologies, Inc.) was cultured in the same medium or a medium further containing keratan sulfate (KS-II: 10 ng/mL), and the culture supernatant was collected. As a control, the cells were cultured in a CpG-free culture medium (NT). The TNF-α amount in each supernatant was measured by ELISA.

The results are shown in FIG. 12. TNF-α production was induced by culturing the cells in a CpG-containing culture medium (CpG). TNF-α production was suppressed when the culture medium was added with KS-II, which is a native DCIR ligand (CpG+KS). In a plurality of hybridoma culture supernatants, production of TNF-α was highly suppressed compared with the case of containing KS-II (3, 4, and 5 strains in FIG. 12). The antibodies derived from these hybridomas have higher DCIR binding abilities than that of KS-II, which is a native DCIR ligand, and are useful as DCIR agonists that enhance DCIR activity. Conversely, in a part of hybridoma culture supernatants, production of TNF-α was promoted compared with the case of containing KS-II (1 and 2 strains in FIG. 12). The antibodies derived from these hybridomas are useful as DCIR antagonists that suppress DCIR activity.

In addition, as shown in the above-described results, substances that can specifically activate or suppress DCIR can be evaluated or selected by using the activity of KS-II, which is a native DCIR ligand, on DCIR as an indicator.

### Example 7

### Influence of Anti-Dcir Antibody on Osteoclast Production

Furthermore, activities of the above-mentioned antibodies on osteoclast formation were evaluated using the activity of keratan sulfate as an indicator. Nonadherent bone marrow cells were seeded in a well plate (a 96-well plate at 1×10⁵ cells/well) and were cultured in α-MEM (Gibco BRL) containing 10% FCS (manufactured by Biowest AG) and 10 ng/mL M-CSF (manufactured by R&D Systems, Inc.). After two days, the nonadherent cells (including lymphocytes) were washed away, and the remaining adherent cells were used as bone marrow-derived macrophages (BMMs). These osteoclast precursor cells (pOCs) were further cultured in the presence of 100 ng/mL of soluble RANKL (manufactured by PeproTech Inc. or Oriental Yeast Co., Ltd.) and 10 ng/mL of M-CSF to obtain osteoclasts. During the culturing, the hybridoma supernatant (one-tenth amount) or keratan sulfate (KS-II: 10 ng/mL) was added to the culture medium. After three days, the cells were collected and stained with TRAP. The number of TRAP-positive osteoclasts (having three or more nuclei) was counted. Untreated cells were used as a control (NT).

The results are shown in FIG. 13. Addition of KS-II, which is a native DCIR ligand, suppressed differentiation to osteoclast (KS). The hybridoma supernatants (DCIR agonist (3, 4, 5 strains in FIG. 13)) showing higher DCIR binding abilities than that of keratan sulfate in Example 5 suppressed osteoclast differentiation, compared with keratan sulfate. The hybridoma supernatants (1 and 2 strains in FIG. 13) that showed DCIR antagonistic activities in Example 5 promoted osteoclast differentiation, compared with keratan sulfate.

These results show that DCIR agonists such as antibodies derived from hybridomas of the present invention increase bone volumes by suppressing osteoclast differentiation and are useful for treatment of osteopenic diseases such as osteoporosis or bone disorders such as fractures. In addition, it is suggested that DCIR antagonists such as antibodies derived from other hybridomas of the present invention regulate bone volumes by promoting osteoclast differentiation and are useful for treatment of various bone diseases.

### Test Example

Effects on collagen-induced arthritis can be confirmed as follows:

Material and method:

Mouse: C57BL/6(B6)(H-2^{b})

Collagen-induced arthritis (CIA): Complete Freund's adjuvant (CFA) is prepared by crushing 100 mg of heat-killed M. tuberculosis cells (H37Ra: manufactured by Difco Laboratories, Inc., Detroit, Mich.) in 20 mL of IFA (manufactured by Sigma Chemical Company, St. Louis, Mo.). An emulsion is prepared by dissolving 2 mg/mL chick type II collagen (CII) (manufactured by Sigma Corp.) in 10 mM acetic acid at 4° C. overnight and mixing the solution with an equal volume of CFA. The mixture is emulsified by a syringe-syringe method. The CII solution and its CFA emulsion are constantly newly prepared. Mice are intercutaneously injected with 100 μL of emulsion containing 100 μg of CII and 250 μg of M. tuberculosis in total at several positions of the base near the tail. This injection is repeated for 21 days.

Clinical evaluation of arthritis: Redness and swelling of limbs of animals are evaluated.

1. A dendritic cell immunoreceptor agonist comprising keratan sulfate-II (KS-II) as an active ingredient.

2. A method of screening for a dendritic cell immunoreceptor agonist or a dendritic cell immunoreceptor antagonist, comprising measuring binding affinity of a test substance to a dendritic cell immunoreceptor in the presence of keratan sulfate-II or using keratan sulfate-II as a control.

3. An antibody against a dendritic cell immunoreceptor, having a keratan sulfate-II-like dendritic cell immunoreceptor agonism.

4. The antibody according to claim 3, wherein the antibody is a monoclonal antibody.

5. The antibody according to claim 3 or 4, having an osteoclast production-suppressing activity and a TNF-α production-suppressing activity.

6. A pharmaceutical comprising the antibody according to claim 3.

7. The pharmaceutical according to claim 6, wherein the pharmaceutical is selected from agents for preventing or treating diseases accompanied by abnormal bone metabolism and agents for preventing or treating inflammatory diseases.

8. An antibody against a dendritic cell immunoreceptor, having a keratan sulfate-II inhibitory dendritic cell immunoreceptor antagonism.

9. The antibody according to claim 8, wherein the antibody is a monoclonal antibody.

10. The antibody according to claim 8 or 9, having an osteoclast production-promoting activity and a TNF-α production-promoting activity.

11. A pharmaceutical comprising the antibody according to claim 8.

12. The pharmaceutical according to claim 11, wherein the pharmaceutical is selected from anticancer agents and immunostimulants.

13. Keratan sulfate-II (KS-II) used for agonizing a dendritic cell immunoreceptor.

14. The antibody according to claim 3, wherein the antibody is used for preventing or treating a disease accompanied by abnormal bone metabolism or an inflammatory disease.

15. The antibody according to claim 8, wherein the antibody is used for treating a cancer or for immunostimulation.

16. A method of agonizing a dendritic cell immunoreceptor, comprising administering keratan sulfate-II (KS-II).

17. A method of preventing or treating a disease accompanied by abnormal bone metabolism or an inflammatory disease, comprising administering the antibody according to claim 3.

18. A method of treating a cancer or of immunostimulation, comprising administering the antibody according to claim 8.


SEQUENCE LISTING (20 sequences)

SEQ ID NO: 1 - DNA, 18 bp, Artificial sequence
  Forward primer for collagen1a1
ggtgcccccggtcttcag

SEQ ID NO: 2 - DNA, 23 bp, Artificial sequence
  Reverse primer for collagen1a1
agggccagggggtccagcatttc

SEQ ID NO: 3 - DNA, 21 bp, Artificial sequence
  Forward primer for osteocalcin
ctgacctcacagatcccaagc

SEQ ID NO: 4 - DNA, 22 bp, Artificial sequence
  Reverse primer for osteocalcin
tggtctgatagctcgtcacaag

SEQ ID NO: 5 - DNA, 22 bp, Artificial sequence
  Forward primer for osteopontin
tagcttggcttatggactgagg

SEQ ID NO: 6 - DNA, 21 bp, Artificial sequence
  Reverse primer for osteopontin
agactcaccgctcttcatgtg

SEQ ID NO: 7 - DNA, 19 bp, Artificial sequence
  Forward primer for bone siaro protein
acaatccgtgccactcact

SEQ ID NO: 8 - DNA, 20 bp, Artificial sequence
  Reverse primer for bone siaro protein
tttcatcgagaaagcacagg

SEQ ID NO: 9 - DNA, 20 bp, Artificial sequence
  Forward primer for ALP
ggacaggacacacacacaca

SEQ ID NO: 10 - DNA, 20 bp, Artificial sequence
  Reverse primer for ALP
caaacaggagagccacttca

SEQ ID NO: 11 - DNA, 21 bp, Artificial sequence
  Forward primer for RunX2
tgttctctgatcgcctcagtg

SEQ ID NO: 12 - DNA, 23 bp, Artificial sequence
  Reverse primer for RunX2
cctgggatctgtaatctgactct

SEQ ID NO: 13 - DNA, 20 bp, Artificial sequence
  Forward primer for Osterix
cccacccttccctcactcat

SEQ ID NO: 14 - DNA, 18 bp, Artificial sequence
  Reverse primer for Osterix
ccttgtaccagccatagg

SEQ ID NO: 15 - DNA, 21 bp, Artificial sequence
  Forward primer for RANKL
cagcatcgctctgttcctgta

SEQ ID NO: 16 - DNA, 21 bp, Artificial sequence
  Reverse primer for RANKL
ctgcgttttcatggagtctca

SEQ ID NO: 17 - DNA, 21 bp, Artificial sequence
  Forward primer for OPG
acccagaaactggtcatcagc

SEQ ID NO: 18 - DNA, 23 bp, Artificial sequence
  Reverse primer for OPG
ctgcaatacacacactcatcact

SEQ ID NO: 19 - DNA, 20 bp, Artificial sequence
  Forward primer for GAPDH
ttcaccaccatggagaaggc

SEQ ID NO: 20 - DNA, 20 bp, Artificial sequence
  Reverse primer for GAPDH
ggcatggactgtggtcatga